(19) United States
(12) Patent Application Publication
(10) Pub. No.: US 2013/0261134 A1
(43) Pub. Date: Oct. 3, 2013

(54) MESYLATE SALT FORMS OF A POTENT HCV INHIBITOR

(71) Applicants: Zhibin LI, Berlin, CT (US); Bing-Shiou YANG, Southbury, CT (US); Kazuhiko YIP, Mystic, CT (US)

(72) Inventors: Zhibin LI, Berlin, CT (US); Bing-Shiou YANG, Southbury, CT (US); Kazuhiko YIP, Mystic, CT (US)

(73) Assignee: Boehringer Ingelheim International GmbH, Ingelheim am Rhein (DE)

(21) Appl. No.: 13/836,644

(22) Filed: Mar. 15, 2013

Related U.S. Application Data
(60) Provisional application No. 61/617,979, filed on Mar. 30, 2012.

(51) Int. Cl. C07D 403/14; A61K 45/06; A61K 31/506

(52) U.S. Cl. CPC C07D 403/14; A61K 31/506; A61K 45/06
USPC 514/256; 544/333; 435/184

(57) ABSTRACT

This invention relates to novel mesylate salt forms of the following Compound (1), and methods for the preparation thereof, pharmaceutical compositions thereof, and their use in methods for the treatment of Hepatitis C Viral (HCV) infection:

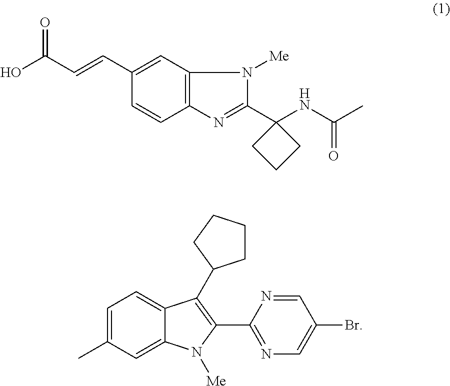

### FIELD OF THE INVENTION

This invention relates to novel solid state forms of Compound (1), including the crystalline form of the mesylate (i.e., methanesulfonate) salt of Compound (1) as described herein, methods for the preparation thereof, pharmaceutical compositions thereof, and their use in the treatment of Hepatitis C Viral (HCV) infection.

### BACKGROUND OF THE INVENTION

The following Compound (1):

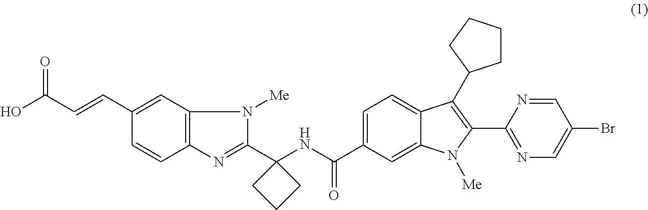 [SMILES: Cn1c(C2(NC(=O)c3ccc4c(C5CCCC5)c(-c5ncc(Br)cn5)n(C)c4c3)CCC2)nc2ccc(/C=C/C(=O)O)cc21]

having the chemical name: (E)-3-[2-(1-{[2-(5-Bromo-pyrimidin-2-yl)-3-cyclopentyl-1-methyl-1H-indole-6-carbonyl]-amino}-cyclobutyl)-3-methyl-3H-benzimidazol-5-yl]-acrylic acid, is known as a selective and potent inhibitor of the HCV NS5B RNA-dependent RNA polymerase and useful in the treatment of HCV infection. Compound (1) falls within the scope of HCV inhibitors disclosed in U.S. Pat. Nos. 7,141,574; 7,582,770; and 7,893,084. Compound (1) is disclosed specifically as Compound #3085 in U.S. Pat. No. 7,582,770. Compound (1), and pharmaceutical formulations thereof, can be prepared according to the general procedures found in the above-cited references, all of which are herein incorporated by reference in their entirety.

When synthesized according to the general procedures set forth in the above-cited references, Compound (1) is prepared as an amorphous solid which is a form that is generally less suitable for full-scale pharmaceutical processing. Thus, there is a need to produce Compound (1) in a form sufficient to enable formulations to meet exacting pharmaceutical requirements and specifications, while providing sufficient in-vivo exposure of the active drug. Furthermore, the process by which Compound (1) is produced needs to be one which is amenable to large-scale production. Additionally, it is desirable that the product should be in a form that is easily processed, e.g. readily filterable and easily dried. Finally, it is economically desirable that the product be stable for extended periods of time without the need for specialized storage conditions.

### SUMMARY OF THE INVENTION

We have now found for the first time that Compound (1) can be prepared in the form of its mesylate salt, and more preferably the crystalline mesylate salt form, and particularly preferably the anhydrous crystalline mesylate form. This novel crystalline form has advantageous properties. For example, the crystalline form of this compound provides solid state stability and may provide advantageous dissolution and solubility characteristics compared to the free acid form. Also, advantages in ease of preparation of the mesylate salt form have also been found, as described below.

Yet another embodiment is directed to pharmaceutical compositions comprising the crystalline Compound (1) mesylate salt, preferably anhydrous crystalline form, and at least one pharmaceutically acceptable carrier or diluent.

Yet another embodiment is directed to a method of treating HCV infection in a mammal comprising administering to said mammal a therapeutically effective amount of the crystalline Compound (1) mesylate salt, preferably anhydrous crystalline form.

### BRIEF DESCRIPTION OF THE DRAWINGS

FIG. 1 is a characteristic X-ray Powder Diffraction (XRPD) pattern for the crystalline mesylate salt of Compound (1).

FIG. 2 shows Solid-state NMR (ssNMR) spectroscopy data for the crystalline mesylate salt of Compound (1).

### DETAILED DESCRIPTION OF THE INVENTION

### Definitions

Terms not specifically defined herein should be given the meanings that would be given to them by one of skill in the art in light of the disclosure and the context. As used throughout the present application, however, unless specified to the contrary, the following terms have the meaning indicated:

The term “about” means within 5%, and more preferably within 1% of a given value or range. For example, “about 3.7%” means from 3.5 to 3.9%, preferably from 3.66 to 3.74%. When the term “about” is associated with a range of values, e.g., “about X % to Y %”, the term “about” is intended to modify both the lower (X) and upper (Y) values of the recited range. For example, “about 20% to 40%” is equivalent to “about 20% to about 40%”.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

### Mesylate Salt of Compound (1)

Compound (1) is a poorly soluble compound with solubility of less than 0.2 μg/mL in the physiological pH range of 2-6.8. Doses of Compound (1) up to 400 to 600 mg per dose may be required to be delivered to obtain exposures necessary for sufficient efficacy in vivo.

The Compound (1) active drug moiety has both acid and basic functional groups which lends itself to salt formation. In general, the conversion of the free form to salt form is known to aid solubilization of poorly water soluble drug substances. Multiple pharmaceutically acceptable acid and basic salt forms of Compound (1) can be produced via crystallization. Methods for preparing the mesylate salt are described herein. The crystalline mesylate salt form is preferred because it provides adequate solid state stability.

Crystalline Mesylate Salt Form

Compound (1) mesylate salt was discovered only after an extensive solid form and polymorph screen conducted using 15 counter ions, 12 solvents and their combinations.

The present invention provides a process for the preparation of crystalline mesylate salt of Compound (1) which comprises crystallizing Compound (1) from a solution in solvents under conditions which yield crystalline mesylate salt. The precise conditions under which crystalline mesylate salt is formed may be empirically determined and it is possible to give methods which have been found to be suitable in practice, as described hereinbelow.

One example of a process that has been found suitable to prepare crystalline mesylate salt is as follows:
(a) Combining Compound (1) with a suitable solvent, such as ethyl acetate, MeCN, MEK or THF, and an aqueous methanesulfonic acid solution, with heating to a temperature of about 50-60° C.;
(b) Stirring and optionally adding additional solvent to the mixture obtained in step (a) at about 50-60° C.;
(c) Cooling the mixture obtained in step (b) to about 20-30° C., resulting in precipitation of Compound (1) mesylate salt crystals; and
(d) Filtering with optional solvent wash and drying.

In step (a), Compound (1) can be combined with the solvent first and this combination can be heated starting either before or after addition of the methanesulfonic acid solution. Alternatively, Compound (1) can be combined with the solvent and methanesulfonic acid solution and then this combination heated. All of these variations are considered covered by the step (a) as set forth above.

Specific procedures found to be suitable for preparing crystalline Compound (1) mesylate salt, as well as formulations that may be prepared using the crystalline mesylate salt, are as described herein. The process for preparation of the mesylate salt is advantageous in that a solvate form is not prepared in this process and thus extra processing for a solvate is avoided. The prepared crystalline form of Compound (1) mesylate salt can either be used directly as it is or subject to an appropriate process to (1) reduce the extent of agglomeration of drug substance particles and/or (2) reduce the particle size distribution of the drug substance primary particles. The process used can include sieving, deagglomeration, impact milling, jet milling or combinations thereof. Details on the use of crystalline Compound (1) mesylate salt in various solid dosage formulation compositions are discussed herein.

In one aspect, the present invention is directed to the crystalline mesylate salt of Compound (1). This crystalline mesylate salt of the Compound (1) has been found to be especially suitable for pharmaceutical processing due to the fact that it can be prepared as a stable crystalline form with advantageous properties as described herein.

The crystalline mesylate salt has been characterized using X-Ray Powder Diffractometry (XRPD) and Solid State NMR (ssNMR). These characterization methods and the results thereof are described below.

X-Ray Powder Diffraction (XRPD)

X-ray powder diffraction analyses were conducted on a Bruker AXS X-Ray Powder Diffractometer Model D8 Advance (Bruker AXS, Inc., Madison, Wis.) using CuKα radiation (1.54 Å). The instrument is equipped with a long fine focus x-ray tube. The tube power was set to 40 kV and 40 mA. The instrument was operated in parafocusing mode using a 0.6 mm divergence slit and a 3.0 mm antiscatter slit. A LynxEye Position Sensitive detector was used to collect the diffraction pattern. Step scans were run from 2 to 35° (degrees 2θ), at 0.015° per step, 0.5 sec per step. A reference quartz standard was used to check instrument alignment. Samples were prepared for analysis by filing a zero background silicon holder. The XRPD analyses were conducted under ambient laboratory conditions, 25° C./25% RH.

The crystalline mesylate salt of Compound (1) exhibits a characteristic X-ray powder diffraction (XRPD) pattern with primary characteristic peaks expressed in degrees 2θ (±0.2 degrees 2θ) at 9.2, 16.9, 21.8, and 23.6. These four XRPD peaks are believed to be sufficient to uniquely identify the presence of the crystalline form of Compound (1) mesylate salt. Further, characteristic peaks are, expressed in degrees 2θ (±0.2 degrees 2θ), at 6.7, 8.8, 9.6, 15.0, 16.6, 19.5 and 20.9.

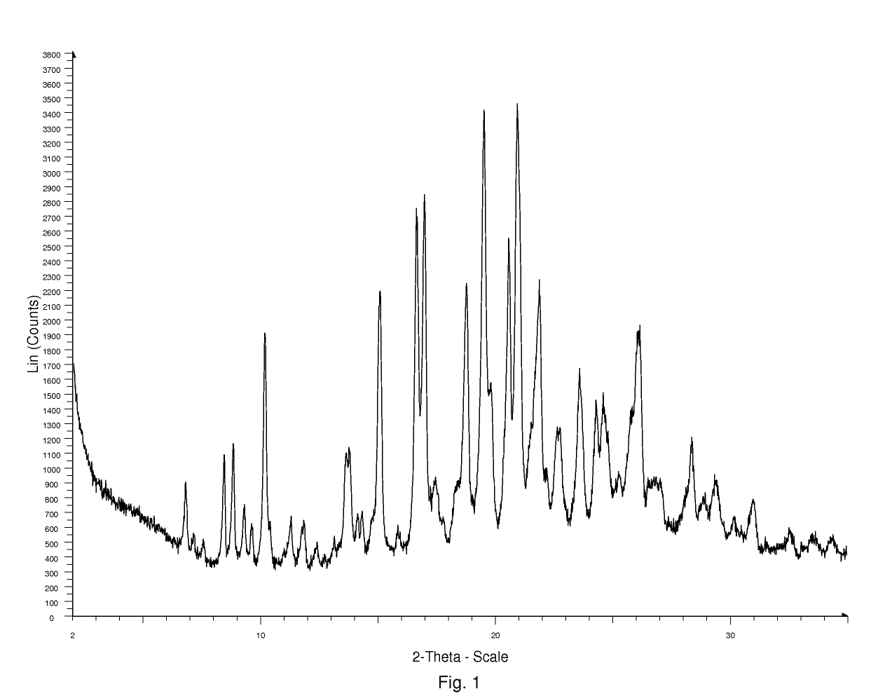

The XRPD pattern of the crystalline mesylate salt of Compound (1) is shown in FIG. 1.

In a general embodiment, the present invention is directed to a crystalline mesylate salt of Compound (1) that has at least the following characteristics: an X-ray powder diffraction pattern comprising peaks at 9.2, 16.9, 21.8, and 23.6 degrees 2θ (±0.2 degrees 2θ) when measured using CuKα radiation. In another embodiment, the present invention is directed to a crystalline mesylate salt of Compound (1) that has at least the following characteristics: an X-ray powder diffraction pattern comprising peaks at 9.2, 16.9, 21.8, and 23.6 degrees 2θ (±0.2 degrees 2θ) and at 6.7, 8.8, 9.6, 15.0, 16.6, 19.5 and 20.9 degrees 2θ (±0.2 degrees 2θ) when measured using CuKα radiation.

The error range of ±0.2 degrees 2θ as stated herein for the various XRPD embodiments applies to all the listed peaks.

Another embodiment is directed to the crystalline mesylate salt of Compound (1) exhibiting an XRPD pattern substantially the same as that shown in FIG. 1.

Solid State NMR (ssNMR) Spectroscopy

Solid-state NMR (ssNMR) data was acquired on a Bruker Avance III NMR spectrometer (Bruker Biospin, Inc., Billerica, Mass.) at 9.4T (¹H=400.46 MHz, ¹³C=100.70 MHz). Samples were packed in 4 mm 0 D zirconia rotors with Kel-F® drive tips. A Bruker model 4BL CP BB WVT probe was used for data acquisition and sample spinning about the magic-angle (54.74°). Sample spectrum acquisition used a spinning rate of 14 kHz. A standard cross-polarization pulse sequence was used with a ramped Hartman-Hahn match pulse on the proton channel at ambient temperature and pressure. The pulse sequence used a 2 millisecond contact pulse and a 5 second recycle delay. Two-pulse phase modulated (tppm) decoupling was also employed in the pulse sequence. No exponential line broadening was used prior to Fourier transformation of the free incution decay. Chemical shifts were referenced using the secondary standard of adamantane, with the upfield resonance being set to 29.5 ppm. The magic-angle was set using the ⁷⁹Br signal from KBr powder at a spinning rate of 5 kHz.

The ¹³C chemical shifts for crystalline Compound (1) mesylate salt are reported in Table 1 below.

TABLE 1
Chemical Shifts
(ppm)

168.5
167.4
159.9
158.3
156.9
155.4
145.8
143.8
140.2
138.6
135.2
133.5
131.9
131.1
129.0
127.5
126.3
123.7
122.0
121.1
118.5
117.0
114.2
112.0
110.9
55.6
54.9
39.2
37.7
36.6
35.7
35.0
34.4
33.7
33.1
31.1
30.2
28.4
26.3
25.5
19.9

The chemical shifts reported and claimed herein are accurate to within ±0.2 ppm unless otherwise indicated.

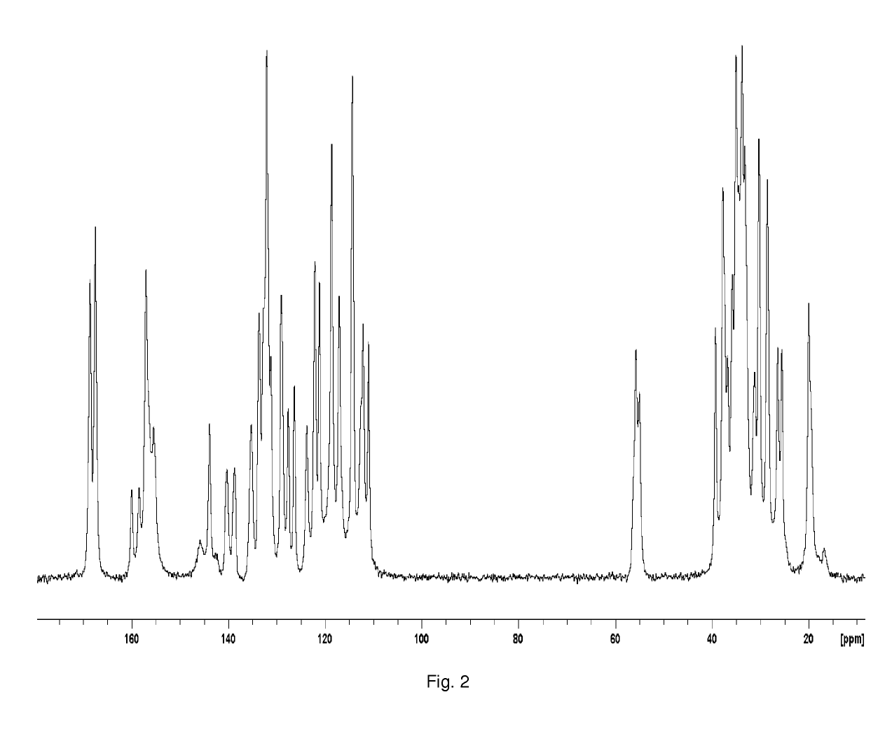

A representative ¹³C ssNMR spectrum of Compound (1) mesylate salt is shown in FIG. 2.

One general embodiment is directed to a crystalline mesylate salt of Compound (1) that has a ¹³C solid state NMR spectrum comprising peaks at chemical shifts of 167.4, 114.2 and 19.9 ppm (±0.2 ppm). These NMR peaks are believed to be sufficient to uniquely identify the presence of the crystalline form of Compound (1) mesylate salt.

Another embodiment is directed to a crystalline mesylate salt of Compound (1) that has ¹³C solid state NMR spectrum comprising peaks at chemical shifts of 167.4, 114.2 and 19.9 ppm and further comprising peaks at chemical shifts of 135.2, 112.0, 39.2 and 28.4 ppm (±0.2 ppm).

Another embodiment is directed to a crystalline mesylate salt of Compound (1) that has additional ¹³C solid state NMR spectrum comprising peaks at chemical shifts of 167.4, 114.2, 19.9, 135.2, 112.0, 39.2 and 28.4 and further comprising peaks at chemical shifts of 133.5, 123.7, 35.7 ppm (±0.2 ppm).

The error range of ±0.2 ppm as stated herein for the various ssNMR embodiments applies to all the listed peaks.

Another embodiment is directed to the crystalline mesylate salt of Compound (1) exhibiting a ¹³C ssNMR spectrum substantially the same as that shown in FIG. 2.

All of the solid state NMR embodiments and corresponding claimed embodiments as set forth herein represent the solid state NMR of the crystalline mesylate salt of Compound (1) when conducted under ambient laboratory conditions (temperature 17-25° C.; relative humidity 30-60%).

Additional XRPD and NMR Embodiments

Additional embodiment are directed to a crystalline mesylate salt of Compound (1) having any combination of the above-disclosed XRPD and ssNMR embodiments.

For example, one embodiment is directed to a crystalline mesylate salt of Compound (1) having an X-ray powder diffraction pattern comprising peaks at 9.2, 16.9, 21.8, and 23.6 degrees 2θ (±0.2 degrees 2θ) when measured using CuKα radiation and a ¹³C solid state NMR spectrum comprising peaks at chemical shifts of 167.4, 114.2, 19.9 ppm (±0.2 ppm).

In an additional embodiment, the crystalline mesylate salt has an X-ray powder diffraction pattern comprising peaks at 9.2, 16.9, 21.8, and 23.6 degrees 2θ (±0.2 degrees 2θ) when measured using CuKα radiation and having a ¹³C solid state NMR spectrum comprising peaks at chemical shifts of 167.4, 114.2, 19.9 ppm and at 135.2, 112.0, 39.2, 28.4 ppm (±0.2 ppm).

In an further additional embodiment, the crystalline mesylate salt has an X-ray powder diffraction pattern comprising peaks at 9.2, 16.9, 21.8, and 23.6 degrees 2θ (±0.2 degrees 2θ) when measured using CuKα radiation and having a ¹³C solid state NMR spectrum comprising peaks at chemical shifts of 167.4, 114.2, 19.9 ppm and at 135.2, 112.0, 39.2, 28.4 ppm and at 133.5, 123.7, 35.7 ppm (±0.2 ppm).

Another embodiment is directed to a crystalline mesylate salt of Compound (1) having an X-ray powder diffraction pattern comprising peaks at 9.2, 16.9, 21.8, and 23.6 degrees 2θ and at 6.7, 8.8, 9.6, 15.0, 16.6, 19.5 and 20.9 degrees 2θ (±0.2 degrees 2θ) when measured using CuKα radiation and a ¹³C solid state NMR spectrum comprising peaks at chemical shifts of 167.4, 114.2, 19.9 ppm (±0.2 ppm).

In another embodiment, the crystalline mesylate salt has an X-ray powder diffraction pattern comprising peaks at 9.2, 16.9, 21.8, and 23.6 degrees 2θ and at 6.7, 8.8, 9.6, 15.0, 16.6, 19.5 and 20.9 degrees 2θ (±0.2 degrees 2θ) when measured using CuKα radiation and having a ¹³C solid state NMR spectrum comprising peaks at chemical shifts of 167.4, 114.2, 19.9 ppm and at 135.2, 112.0, 39.2, 28.4 ppm (±0.2 ppm).

In another embodiment, the crystalline mesylate salt has an X-ray powder diffraction pattern comprising peaks at 9.2, 16.9, 21.8, and 23.6 degrees 2θ and at 6.7, 8.8, 9.6, 15.0, 16.6, 19.5 and 20.9 degrees 2θ (±0.2 degrees 2θ) when measured using CuKα radiation and having a ¹³C solid state NMR spectrum comprising peaks at chemical shifts of 167.4, 114.2, 19.9 ppm and at 135.2, 112.0, 39.2, 28.4 ppm and at 133.5, 123.7, 35.7 ppm (±0.2 ppm).

Additional peak combinations are, of course, possible and are contemplated herein.

### Additional Embodiments

Another embodiment is directed to crystalline mesylate salt of Compound (1), wherein said crystalline mesylate salt is substantially pure as defined herein.

The term “substantially pure” when referring to a designated crystalline form of Compound (1) mesylate salt means that the designated crystalline form contains less than 20% (by weight) of residual components such as alternate polymorphic or isomorphic crystalline form(s) thereof, or alternative salt forms thereof. It is preferred that a substantially pure form of Compound (1) mesylate salt contain less than 10% (by weight) of alternate polymorphic or isomorphic crystalline forms, more preferred is less than 5% (by weight) of alternate polymorphic or isomorphic crystalline forms, and most preferably less than 1% (by weight) of alternate polymorphic or isomorphic crystalline forms.

Another embodiment is therefore directed to crystalline mesylate salt of Compound (1) being in substantially pure form, i.e., wherein at least 80%, preferably at least 90%, more preferably at least 95%, more preferably at least 99%, of said substance is present in the form of crystalline mesylate salt of Compound (1), as may be characterized by any of the abovementioned XRPD or ssNMR embodiments.

An additional embodiment is directed to a pharmaceutical composition comprising crystalline Compound (1) mesylate salt and at least one pharmaceutically acceptable carrier or diluent. In a more specific embodiment, the crystalline Compound (1) mesylate salt in the pharmaceutical composition is as defined by any of the above-mentioned XRPD and/or ssNMR embodiments. In further specific embodiment, the crystalline Compound (1) mesylate salt is substantially pure as defined by any of the above-mentioned XRPD and/or ssNMR embodiments. That is, at least 80%, preferably at least 90%, more preferably at least 95%, more preferably at least 99%, of the Compound (1) mesylate salt in the composition is present in crystalline form, as characterized by any of the abovementioned XRPD and/or ssNMR embodiments.

The XRPD and/or ssNMR characterization methods set forth herein can be used to quantify the relative amounts of the preferred crystalline mesylate salt form of Compound (1) present in the material.

Pharmaceutical Compositions and Methods

The mesylate salt forms of Compound (1) described herein are useful as anti-HCV agents in view of the demonstrated inhibitory activity of Compound (1) against HCV NS5B RNA-dependent RNA polymerase. The form is therefore useful in treatment of HCV infection in a mammal and can be used for the preparation of a pharmaceutical composition for treating an HCV infection or alleviating one or more symptoms thereof in a patient. The appropriate dosage amounts and regimens for a particular patient can be determined by methods known in the art and by reference to the disclosure in U.S. Pat. Nos. 7,141,574 and 7,582,770, and 7,893,084. Generally, a therapeutically effective amount for the treatment of HCV infection in the mammal is administered. In one embodiment, about 1200 mg to 1800 mg is administered per adult human per day in single or multiple doses.

Specific optimal dosage and treatment regimens for any particular patient will of course depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

The mesylate salt form of Compound (1) at a selected dosage level is typically administered to the patient via a pharmaceutical composition. See, for example, the descriptions in U.S. Pat. Nos. 7,141,574 and 7,582,770, and 7,893,084 for the various types of compositions that may be employed in the present invention. The pharmaceutical composition may be administered orally, parenterally, topically or via an implanted reservoir, for example. The term parenteral as used herein includes subcutaneous, intracutaneous, intravenous, intramuscular, intra-articular, intrasynovial, intrasternal, intrathecal, and intralesional injection or infusion techniques. Oral administration is preferred.

The pharmaceutical compositions of this invention may contain any conventional non-toxic pharmaceutically-acceptable carriers, diluents, adjuvants, excipients or vehicles. In some cases, the pH of the formulation may be adjusted with pharmaceutically acceptable acids, bases or buffers to enhance the stability of the formulated compound or its delivery form.

The pharmaceutical compositions may also be in the form of an oral pharmaceutical composition comprising the crystalline mesylate salt of Compound (1) and at least one pharmaceutically acceptable carrier or diluent. The oral pharmaceutical compositions may be orally administered in any orally acceptable dosage form including, but not limited to, tablets, capsules (e.g., hard or soft gelatin capsules), including liquid-filled capsules, and aqueous suspensions and solutions. In the case of tablets or extrudates casted into tablets for oral use, carriers which are commonly used include lactose, mannitol, sugars and corn starch. Lubricating agents, such as magnesium stearate, are also typically added. For oral administration in a capsule form, useful diluents include lactose, mannitol, sugars, microcrystalline cellulose and cellulose derivatives and dried corn starch. Examples of soft gelatin capsules that can be used include those disclosed in EP 649651 B1 and U.S. Pat. No. 5,985,321. When aqueous suspensions are administered orally, the active ingredient is combined with emulsifying and suspending agents. If desired, certain sweetening and/or flavoring and/or coloring agents may be added. Other suitable vehicles or carriers for the above noted formulations and compositions can be found in standard pharmaceutical texts, e.g. in “Remington's Pharmaceutical Sciences”, 19^{th} ed., Mack Publishing Company, Easton, Pa., 1995.

Certainly, when the crystalline mesylate salt is formulated in a liquid vehicle, for example, as a liquid solution or suspension for oral administration or by injection, including for example in liquid-filled capsules, the mesylate salt loses its crystalline nature. Nevertheless, the final liquid-based pharmaceutical composition contains the novel mesylate salt of Compound (1) and it is therefore to be considered a separate embodiment embraced by the present invention. It was only by discovering a method for preparing the mesylate salt in a stable crystalline form that the present inventors enabled efficient pharmaceutical processing and pharmaceutical formulation manufacture using the mesylate salt form. Therefore, the final pharmaceutical formulation containing the mesylate salt form which was thereby enabled by this discovery is considered another aspect and embodiment of the present invention.

For oral administration, the compound or a therapeutically acceptable salt thereof can be formulated in unit dosage forms such as capsules or tablets each containing a predetermined amount of the active ingredient, ranging from about 1 to about 500 mg, in a pharmaceutically acceptable carrier.

For topical administration, the compound can be formulated in pharmaceutically accepted vehicles containing about 0.1 to about 5 percent, preferably about 0.5 to about 5 percent, of the active agent. Such formulations can be in the form of a solution, cream or lotion.

For systemic administration, the compound of formula (I) can be administered by either intravenous, subcutaneous or intramuscular injection, in compositions with pharmaceutically acceptable vehicles or carriers. For administration by injection, it is preferred to use the compounds in solution in a sterile aqueous vehicle which may also contain other solutes such as buffers or preservatives as well as sufficient quantities of pharmaceutically acceptable salts or of glucose to make the solution isotonic.

For oral administration, the compound or a therapeutically acceptable salt can be administered in the range of about 0.01 to about 200 mg per kilogram of body weight per day, with a preferred range of about 0.05 to about 100 mg per kilogram.

For systemic administration, the compound of formula (I) can be administered at a dosage of about 0.01 mg to about 100 mg per kilogram of body weight per day, although the aforementioned variations will occur. A dosage level that is in the range of from about 0.05 mg to about 50 mg per kilogram of body weight per day is most desirably employed in order to achieve effective results.

According to another embodiment, the pharmaceutical composition of this invention additionally comprises a therapeutically effective amount of one or more other antiviral agents. Examples of other antiviral agents include, but are not limited to, ribavirin and amantadine.

In another embodiment, the pharmaceutical composition of this invention additionally comprises at least one other anti-HCV agent as an antiviral agent.

In another embodiment, the pharmaceutical composition of this invention comprises an additional immunomodulatory agent as another anti-HCV agent. Examples of additional immunomodulatory agents include but are not limited to, alpha-, beta-, delta-gamma-, tau- and omega-interferons and pegylated forms thereof.

In another embodiment, the pharmaceutical composition of this invention additionally comprises at least one other inhibitor of HCV polymerase as an anti-HCV agent.

In another embodiment, the pharmaceutical composition of this invention additionally comprises at least one inhibitor of HCV NS3 protease as another anti-HCV agent.

In another embodiment, the pharmaceutical composition of this invention additionally comprises at least one inhibitor of another target in the HCV life cycle as an other anti-HCV agent. Examples of such inhibitors of other targets include, but are not limited to, agents that inhibit a target selected from HCV helicase, HCV NS2/3 protease and HCV IRES and agents that interfere with the function of other viral targets including but not limited to an NS5A protein.

As discussed above, combination therapy is contemplated wherein the mesylate salt of Compound (1), is co-administered with at least one additional agent selected from, for example: an antiviral agent, an immunomodulatory agent, an inhibitor of HCV NS3 protease, another inhibitor of HCV polymerase, an inhibitor of another target in the HCV life cycle, an HIV inhibitor, an HAV inhibitor and an HBV inhibitor. Specific preferred examples of such agents are: antiviral agents: ribavirin or amantadine; immunomodulatory agents: class I interferons, class II interferons or pegylated forms thereof; HCV NS3 protease inhibitors; other inhibitors of the HCV polymerase: nucleoside or non-nucleoside inhibitors; an inhibitor of another target in the HCV life cycle that inhibits a target selected from: NS3 helicase, HCV NS2/3 protease and internal ribosome entry site (IRES) or an agent that interferes with the function of an NS5A protein; HIV inhibitors: nucleoside inhibitors, non-nucleoside inhibitors, protease inhibitors, fusion inhibitors or integrase inhibitors; or HBV inhibitors: agents that inhibit HBV viral DNA polymerase or an agent that is an HBV vaccine.

When the compositions of this invention comprise a combination of the mesylate salt of Compound (1) and one or more additional agents, as described above, both the mesylate salt of Compound (1) and the additional agent should be present at dosage levels of between about 10 to 100%, and more preferably between about 10 and 80% of the dosage normally administered in a monotherapy regimen.

The above-described compounds or compositions may be administered in vivo to mammals, such as man, to inhibit HCV polymerase or to treat or prevent HCV virus infection. Such treatment may also be achieved using the mesylate salt of Compound (1) in combination with other agents, such as described above. The additional agents may be combined with the mesylate salt of Compound (1) to create a single dosage form. Alternatively these additional agents may be separately administered to a mammal as part of a multiple dosage form. Such additional agents may be administered to the patient prior to, concurrently with, or following the administration of the mesylate salt of Compound (1).

Another aspect of this invention relates to an article of manufacture, for example a kit, comprising one of the compositions described above containing the methanesulfonate salt of Compound (1) in a form effective to treat or prevent an HCV infection or to inhibit the NS5B polymerase of HCV and packaging material comprising a label which indicates that the composition can be used to treat infection by the hepatitis C virus.

In order that this invention is more fully understood, the following examples are set forth. These examples are for the purpose of illustrating embodiments of this invention, and are not to be construed as limiting the scope of the invention in any way. The reactants used in the examples below may be obtained either as described herein, or if not described herein, are themselves either commercially available or may be prepared from commercially available materials by methods known in the art.

Unless otherwise specified, solvents, temperatures, pressures, and other reaction conditions may be readily selected by one of ordinary skill in the art. Typically, reaction progress may be monitored by High Pressure Liquid Chromatography (HPLC), if desired, and intermediates and products may be purified by chromatography on silica gel and/or by recrystallization.

### EXAMPLES

### Example 1

### Preparation of Compound (1)

### Step 1. Synthesis of Isopropyl 3-Cyclopentyl-1-methyl-1H-indole-6-carboxylate

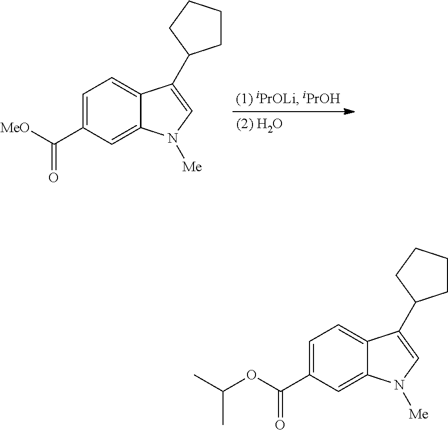 [SMILES: COC(=O)c1ccc2c(C3CCCC3)cn(C)c2c1 ; CC(C)OC(=O)c1ccc2c(C3CCCC3)cn(C)c2c1]

Because of the instability of brominated product, methyl 3-cyclopentyl-1-methyl-1H-indole-6-carboxylate needed to be converted into the more stable isopropyl 3-cyclopentyl-1-methyl-1H-indole-6-carboxylate via a simple and high yielding operation. The conversion worked the best with stoichiometric amounts of solid lithium isopropoxide. Use of 0.1 eq lithium isopropoxide led to longer reaction times and as a result to more hydrolysis by-product, while lithium isopropoxide solution in THF caused a problematic isolation and required distillation of THF.

Procedure:

The mixture of methyl 3-cyclopentyl-1-methyl-1H-indole-6-carboxylate (50.0 g, 0.194 mol) and lithium isopropoxide (16.2 g, 95%, 0.233 mol) in 2-propanol was stirred at 65±5° C. for at least 30 min for complete trans-esterification. The batch was cooled to 40±5° C. and water (600 g) was added at a rate to maintain the batch temperature at 40±5° C. After addition, the mixture was cooled to 20-25° C. over 2±0.5 h and held at 20-25° C. for at least 1 h. The batch was filtered and rinsed with 28 wt % 2-propanol in water (186 g), and water (500 g). The wet cake was dried in vacuo (≦200 Torr) at 40-45° C. until the water content was ≦0.5% to give isopropyl 3-cyclopentyl-1-methyl-1H-indole-6-carboxylate (52.7 g, 95% yield) in 99.2 A % (240 nm).

The starting material methyl 3-cyclopentyl-1-methyl-1H-indole-6-carboxylate can be prepared as described in Example 12 of U.S. Pat. No. 7,141,574, and in Example 12 of U.S. Pat. No. 7,642,352, both herein incorporated by reference.

### Step 2. Synthesis of Isopropyl 2-Bromo-3-cyclopentyl-1-methyl-1H-indole-6-carboxylate

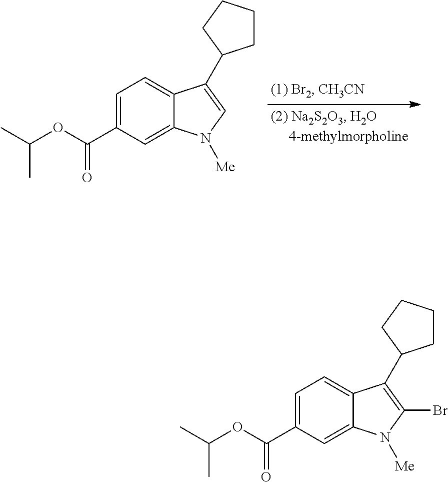 [SMILES: CC(C)OC(=O)c1ccc2c(C3CCCC3)cn(C)c2c1 ; CC(C)OC(=O)c1ccc2c(C3CCCC3)c(Br)n(C)c2c1]

This process identified the optimal conditions for the synthesis of 2-bromo-3-cyclopentyl-1-methyl-1H-indole-6-carboxylate via bromination of the corresponding 3-cyclopentyl-1-methyl-1H-indole-6-carboxylate with bromine. It's very important to control the reaction temperature and to quench the reaction mixture with a mixture of aqueous mesylate thiosulfate and 4-methylmorpholine to minimize the formation of the dibromo- and 2-indolone impurities. Further neutralization of the crude product with NaOH in isopropanol greatly increases the stability of the isolated product.

Procedure:

The mixture of isopropyl 3-cyclopentyl-1-methyl-1H-indole-6-carboxylate (50.0 g, 0.175 mol) and acetonitrile (393 g) was cooled to −6±3° C. Bromine (33.6 g, 0.210 mol) was added while the batch was maintained at −6±3° C. The resulting slurry was stirred at −6±3° C. for at least 30 min When HPLC showed ≧94% conversion (the HPLC sample must be quenched immediately with aqueous 4-methylmorpholine/mesylate thiosulfate solution), the mixture was quenched with a solution of mesylate thiosulfate (15.3 g) and 28.4 g 4-methylmorpholine in water (440 g) while the temperature was maintained at −5±5° C. After it was stirred at 0±5° C. for at least 2 h, the batch was filtered and rinsed with 85 wt % methanol/water solution (415 g), followed by water (500 g), and dried until water content is ≦30%. The wet cake was suspended in 2-propanol (675 g), and heated to 75±5° C. The resulting hazy solution was treated with 1.0 M aqueous mesylate hydroxide solution (9.1 g) and then with 135.0 g water at a rate to maintain the batch at 75±5° C. The suspension was stirred at 75±5° C. for at least 30 min, cooled to 15±2° C. over 30-40 min, and held at 15±2° C. for at least 1 h. The batch was filtered, rinsed with 75 wt % 2-propanol/water solution (161 g), and dried in vacuo (≦200 Torr) at 50-60° C. until the water content was ≦0.4% to give isopropyl 2-bromo-3-cyclopentyl-1-methyl-1H-indole-6-carboxylate as a solid (55.6 g, 87% yield) in 99.5 A % (240 nm) and 97.9 Wt %.

Alternative Procedure:

The mixture of isopropyl 3-cyclopentyl-1-methyl-1H-indole-6-carboxylate (84 g, 0.294 mol) and isopropyl acetate (1074 g) was cooled to between −10-0° C. Bromine (50 g, 0.312 mol) was added while the batch was maintained at −10-0° C. The resulting slurry was stirred at the same temperature for additional 30 min and quenched with a pre-cooled solution of mesylate thiosulfate pentahydrate (13 g) and triethylamine (64.5 g) in water (240 g) while the temperature was maintained at 0-10° C. The mixture was heated to 40-50° C. and charged with methanol (664 g). After it was stirred at the same temperature for at least 0.5 h, the batch was cooled to 0-10° C. and stirred for another 1 hr. The precipitate was filtered, rinsed with 56 wt % methanol/water solution (322 g), and dried in vacuo (≦200 Torr) at 50-60° C. until the water content was ≦0.4% to give isopropyl 2-bromo-3-cyclopentyl-1-methyl-1H-indole-6-carboxylate as a beige solid (90-95 g, 80-85% yield).

### Step 3a,b. Preparation of Compound I by One-Pot Pd-Catalyzed Borylation-Suzuki Coupling Reaction

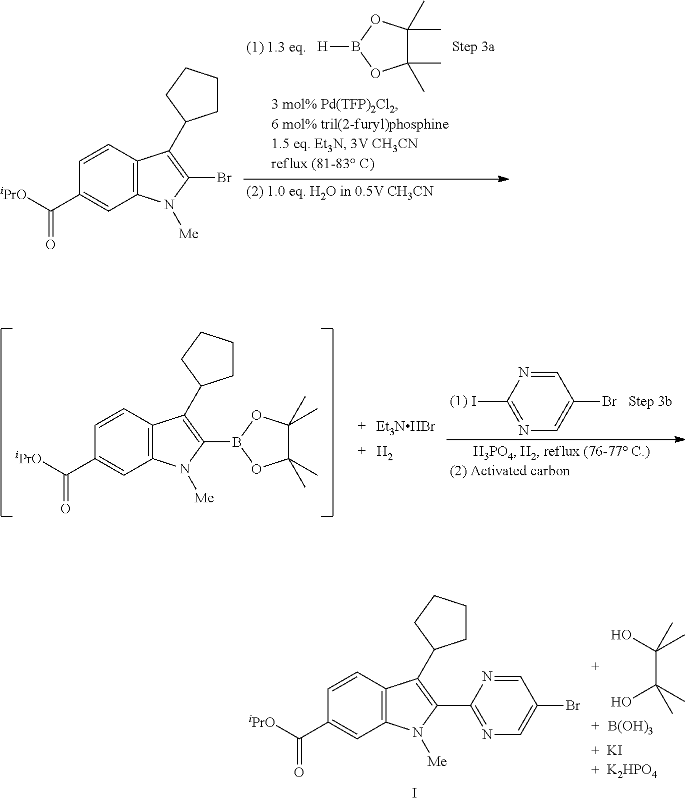

To a clean and dry reactor containing 20.04 g of isopropyl 2-bromo-3-cyclopentyl-1-methyl-1H-indole-6-carboxylate, 1.06 g of Pd(TFP)₂Cl₂ (3 mol %) and 0.76 g of tri(2-furyl)phosphine (6 mol %) was charged 8.35 g of triethylamine (1.5 equivalent), 39.38 g of CH₃CN at 23±10° C. under nitrogen or argon and started agitation for 10 min 9.24 g of 4,4,5,5-tetramethyl-1,3,2-dioxaborolane was charged into the reactor. The mixture was heated to reflux (ca. 81-83° C.) and stirred for 6 h until the reaction completed. The batch was cooled to 30±5° C. and quenched with a mixture of 0.99 g of water in 7.86 g of CH₃CN. 17.24 g of 5-bromo-2-iodopyrimidine and 166.7 g of degassed aqueous potassium phosphate solution (pre-prepared from 46.70 g of K₃PO₄ and 120 g of H₂O) was charged subsequently under argon or nitrogen. The content was heated to reflux (ca. 76-77° C.) for 2 h until the reaction completed. 4.5 g of 1-methylimidazole was charged into the reactor at 70° C. The batch was cooled to 20±3° C. over 0.5 h and hold at 20±3° C. for at least 1 h. The solid was collected by filtration. The wet cake was first rinsed with 62.8 g of 2-propanol, followed by 200 g of H₂O. The solid was dried under vacuum at the temperature below 50° C.

Into a dry and clean reactor was charged dried I, 10 wt % Norit SX Ultra and 5 V of THF. The content was heated at 60±5° C. for at least 1 h. After the content was cooled to 35±5° C., the carbon was filtered off and rinsed with 3 V of THF. The filtrate was charged into a clean reactor containing 1-methylimidazole (10 wt % relative to I). After removal of 5 V of THF by distillation, the content was then cooled to 31±2° C. After the agitation rate was adjusted to over 120 rpm, 2.5 V of water was charged over a period of at least 40 minutes while maintaining the content temperature at 31±2° C. After the content was agitated at 31±2° C. for additional 20 min, 9.5 V of water was charged into the reactor over a period of at least 30 minutes at 31±2° C. The batch was then cooled to about 25±3° C. and stirred for additional 30 minutes. The solid was collected and rinsed with 3 V of water. The wet product I was dried under vacuum at the temperature below 50° C. (19.5 g, 95 wt %, 76% yield).

Alternative Procedure:

To a clean and dry reactor containing 40 g of isopropyl 2-bromo-3-cyclopentyl-1-methyl-1H-indole-6-carboxylate (0.110 mol), 0.74 g of Pd(OAc)₂ (3.30 mmol, 3 mol % equiv.) and 3.2 g of tri(2-furyl)phosphine (13.78 mmol, 12.5 mol % equiv.) was charged 16.8 g of triethylamine (1.5 equivalent), 100 mL of acetonitrile at 25° C. under nitrogen or argon. 20.8 g of 4,4,5,5-tetramethyl-1,3,2-dioxaborolane was charged into the reactor within 30 min. The mixture was heated to reflux (ca. 81-83° C.) and stirred for over 5 hrs until the reaction completed. The batch was cooled to 20° C. and quenched with a mixture of 2.7 g of water in 50 mL of CH₃CN. The batch was warmed to 30° C., stirred for 1 hr and transferred to a second reactor containing 34.4 g of 5-bromo-2-iodopyrimidine in 100 mL of acetonitrile. The reactor was rinsed with 90 mL of acetonitrile. To the second reactor was charged with degassed aqueous potassium phosphate solution (pre-prepared from 93.2 g of K₃PO₄ and 100 g of H₂O) under argon or nitrogen. The content was heated to reflux (ca. 80° C.) for over 3 h until the reaction completed. 9.2 g of 1-methylimidazole was charged into the reactor at 70° C. and the mixture was stirred for at least 10 min. The aqueous phase was removed after phase separation. 257 g of isopropanol was charged at 70° C. The batch was cooled slowly to 0° C. and hold for at least 1 h. The solid was collected by filtration. The wet cake was rinsed twice with 2-propanol (2×164 g) and dried under vacuum at the temperature below 50° C. to give I as a yellow to brown solid (26 g, 75% yield).

### Step 4. Hydrolysis of I to II

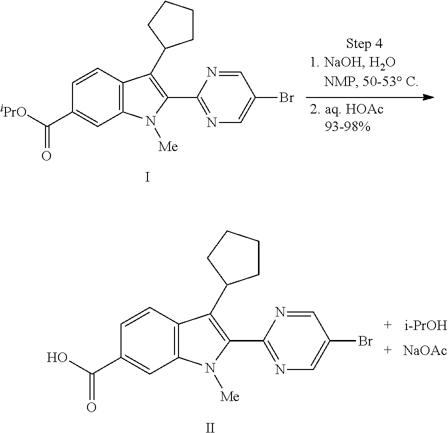

I (20 g) and 1-methyl-2-pyrrolidinone (NMP) (113 g) were charged into a clean reactor under nitrogen. After the batch was heated to 50-53° C. with agitation, premixed aq. NaOH (5.4 g of 50% aq. NaOH and 14.3 g of water) was introduced into the reactor. The resulting mixture was stirred at 50-53° C. for about 10 hrs until the reaction completed. A premixed aq. HOAc (60 g of water and 9.0 g of HOAc) was added over 0.5 h at 45±5° C. to reach pH 5.5-7.5. The batch was cooled to 20±5° C. and then kept for at least 1.0 h. The solid product was collected and rinsed with 80 g of NMP/water (1:3 volume ratio) and then 60 g of water. The product was dried under vacuum at the temperature below 50° C. to give II as a pale yellow powder (19-20 g, purity >99.0 A % and 88.4 wt %, containing 5.4 wt % NMP). The yield is about 93-98%.

Notes: The original procedure used for the hydrolysis of I was carried out with aq. NaOH (2.5 eq) in MeOH/THF at 60° C. Although it has been applied to the preparation of II on several hundred grams scale, one disadvantage of this method is the formation of 5-MeO pyrimidine during hydrolysis (ca. 0.4 A %), which is extremely difficult to remove in the subsequent steps. In addition, careful control has to be exerted during crystallization. Otherwise, a thick slurry might form during acidification with HOAc. The use of NMP as solvent could overcome all aforementioned issues and give the product with desired purity.

Alternative Process

To a reactor was charged I (71 g), isopropanol (332 g), aqueous NaOH (22 g, 45 wt %) and water (140 g) at ambient temperature. The mixture was heated to reflux (80° C.) and stirred for at least 3 hrs until the reaction completed. The batch was cooled to 70° C. and charged a suspension of charcoal (3.7 g) in isopropanol (31 g). The mixture was stirred at the same temperature for over 10 min and filtered. The residue was rinsed with isopropanol (154 g). Water (40 g) was charged to the filtrate at 70-80° C., followed by slow addition of 36% HCl solution (20 g) to reach pH 5-6. The batch was stirred for over 30 min at 70° C., then cooled to 20° C. over 1 hr and kept for at least 1.0 h. The solid product was collected and rinsed with 407 g of isopropanol/water (229 g IPA, 178 g H₂O). The product was dried under vacuum at 80° C. for over 5 hrs to give II as a white powder (61 g, 95% yield).

Notes on Steps 5 to 8 Below:

A concise and scalable 4-step process for the preparation of the benzimidazole intermediate V was developed. The first step was the preparation of 4-chloro-2-(methyl)-aminonitrobenzene starting from 2,4-dichloronitrobenzene using aqueous methyl amine in DMSO at 65° C. Then, a ligandless Heck reaction with n-butyl acrylate in the presence of Pd(OAc)₂, ⁱPr₂NEt, LiCl, and DMAc at 110° C. was discovered.

### Step 5: SNAr reaction of (5-chloro-2-nitrophenyl)-methylamine

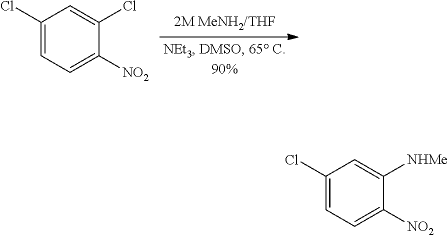 [SMILES: O=[N+]([O-])c1ccc(Cl)cc1Cl ; CNc1cc(Cl)ccc1[N+](=O)[O-]]

To a solution of (5-chloro-2-nitrophenyl)-methylamine (40 g, 208.3 mmol, 1 equiv) in DMSO (160 mL) was added 40% MeNH₂ solution in water (100 mL, 1145.6 mmol, 5.5 eq) slowly keeping the temperature below 35° C. The reaction was stirred at r.t. until the complete consumption of the starting material (>10 h). Water (400 mL) was added to the resulting orange slurry and stirred at r.t. for additional 2 h. The solid was filtered, rinsed with water (200 mL) and dried under reduced pressure at 40° C. (5-chloro-2-nitrophenyl)-methylamine (36.2 g, 93% yield, 94 A % purity) was isolated as a solid.

### Step 6: Heck Reaction of (5-chloro-2-nitrophenyl)-methylamine

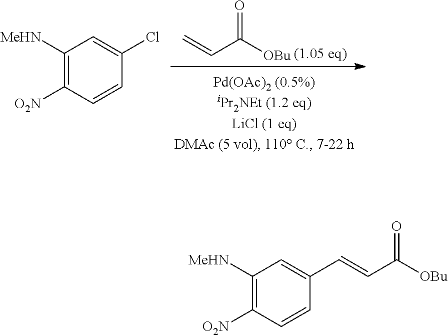

To a mixture of 4-chloro-2-methylaminonitrobenzene (50.0 g, 268.0 mmol, 1.0 eq), Pd(OAc)₂ (0.30 g, 1.3 mmol, 0.005 eq) and LiCl (11.4 g 268.0 mmol, 1.0 eq) in DMAc (250 mL) was added ⁱPr₂NEt (56 mL, 321.5 mmol, 1.2 eq) followed by n-butyl acrylate (40 mL, 281.4 mmol, 1.05 eq) under nitrogen. The reaction mixture was stirred at 110° C. for 12 h, then cooled to 50° C. 1-methylimidazole (10.6 mL, 134.0 mmol, 0.5 eq) was added and the mixture was stirred for 30 min before filtering and adding water (250 mL). The resulting mixture was cooled to r.t. over 1 h. The resulting solid was filtered and washed with water and dried to yield n-butyl 3-methylamino-4-nitrocinnamate (71.8 g, 96%, 99.2 A % purity).

### Step 7: Reduction of n-butyl (3-methylamino-4-nitro)-cinnamate

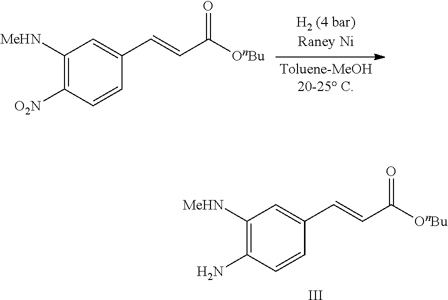

To a reactor was charged n-butyl 3-methylamino-4-nitrocinnamate (70.0 g, mmol, 1.0 eq), Raney Ni (4.9 g, ˜20 wt % H₂O), charcoal “Norit SX Ultra” (3.5 g), toluene (476 mL) and MeOH (224 mL). The reactor was charged with hydrogen (4 bar) and the mixture was stirred at 20-25° C. for about 2 hrs until the reaction was completed. The reaction mixture was filtered and rinsed the filter residue with toluene (70 mL). To the combined filtrates were added “Norit SX Ultra” charcoal (3.5 g). The mixture was stirred at 50° C. for 1.0 hr and filtered. The filtrate was concentrated under reduced pressure to remove solvents to 50% of the original volume. The remained content was heated to 70° C. and charged slowly methyl cyclohexane (335 mL) at the same temperature. The mixture was cooled to about 30-40° C. and seeded with III seed crystals, then slowly cooled the suspension to ˜−10° C. The solid was filtered and rinsed with methyl cyclohexane in three portions (3×46 mL). The wet cake was dried in vacuo at 40° C. to give III (53.3 g, 215 mmol, 86%).

### Step 8: Preparation of Benzimidazole V

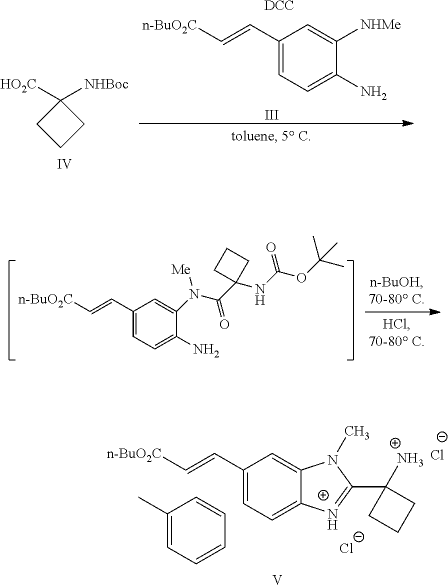

To reactor-1 was charged III (35 g, 140.95 mmol) in toluene (140 g). The mixture was heated to 50° C. to obtain a clear solution. To a second reactor was charged IV (36.4 g, 169.10 mmol) and toluene (300 g), followed by addition of a solution of dicyclohexyl carbodimide (11.6 g, in 50% toluene, 28.11 mmol) at 0-10° C. The mixture was stirred at the same temperature for 15 min, then charged in parallel with the content of reactor-1 and the solution of dicyclohexyl carbodimide (52.4 g, in 50% toluene, 126.98 mmol) within 1 hr while maintaining the batch temperature at 0-10° C. The mixture was agitated at the same temperature for 3 hrs, and warmed to 25° C. for another 1 hr. Once III was consumed, toluene (−300 mL) was distilled off under reduced pressure at 70-80° C. n-Butanol (200 g) was added, followed by 3 M HCl solution in n-butanol (188 g) while maintaining the temperature at 70-80° C. (Gas evolution, product precipitates). After stirring for over 30 min at 70-80° C., the mixture was cooled to 20-30° C. over 1 hr. The precipitate was filtered and washed with acetone (172 g) and toluene (88 g). The wet cake was dried in vacuo at ˜60° C. to give V toluene solvate as off white solid (60-72 g, 85-95% yield). Compound V could be used directly for the next step or basified prior to next step to obtain the free base compound VI used in the next step.

### Step 9. Synthesis of (E)-Butyl 3-(2-(1-(2-(5-Bromopyrimidin-2-yl)-3-cyclopentyl-1-hydroxy-1H-indole-6-carboxamido)cyclobutyl)-1-methyl-1H-benzo[d]imidazol-6-yl)acrylate (VII)

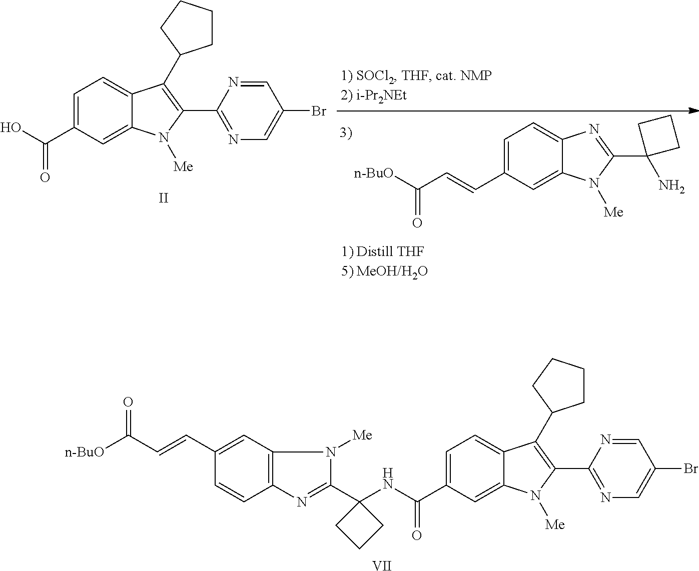

Notes:

The conversion of the acid into acid chloride was achieved using inexpensive thionyl chloride in the presence of catalytic amount of NMP or DMF. An efficient crystallization was developed for the isolation of the desired product in high yield and purity.

Procedure (Using Free Base VI):

To the suspension of 2-(5-bromopyrimidin-2-yl)-3-cyclopentyl-1-methyl-1H-indole-6-carboxylic acid II (see Step 4) (33.36 g, 90.0 wt %, containing ˜0.2 equiv of NMP from previous step, 75.00 mmol) in THF (133.4 g) was added thionyl chloride (10.71 g). The mixture was stirred at 25±5° C. for at least 1 h. After the conversion was completed as determined by HPLC (as derivative of diethylamine), the mixture was cooled to 10±5° C. and N,N-diisopropylethylamine (378.77 g, 300 mmol) below 25° C. A solution of (E)-butyl 3-(2-(1-aminocyclobutyl)-1-methyl-1H-benzo[d]imidazol-6-yl)acrylate VI (25.86 g, 97.8 Wt %, 77.25 mmol) dissolved in THF (106.7 g) was added at a rate to maintain the temperature of the content ≦25° C. The mixture was stirred at 25±5° C. for at least 30 min for completion of the amide formation. The mixture was distilled at normal pressure to remove ca. 197 mL (171.5 g) of volatiles (Note: the distillation can also be done under reduced pressure). The batch was adjusted to 40±5° C., and MeOH (118.6 g) was added. Water (15.0 g) was added and the mixture was stirred at 40±5° C. until crystallization occurred (typically in 30 min), and held for another 1 h. Water (90 g) was charged at 40±5° C. over 1 h, and the batch was cooled to 25±5° C. in 0.5 h, and held for at least 1 h. The solid was filtered, rinsed with a mixture of MeOH (39.5 g), water (100 g), and dried in vacuo (≦200 Torr) at 50±5° C. to give (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate VII (51.82 g, 96.6% yield) with a HPLC purity of 98.0 A % (240 nm) and 99.0 Wt %.

Alternative Procedure (Using Compound V from Step 8)

To reactor 1 was charged 2-(5-bromopyrimidin-2-yl)-3-cyclopentyl-1-methyl-1H-indole-6-carboxylic acid II (33.6 g), toluene (214 g) and N-methylpyrrolidone (1.37 g). The mixture was heated to 40° C., then added a solution of thionyl chloride (13 g) in toluene (17 g). The mixture was stirred at 40° C. for at least 0.5 h and cooled to 30° C. To a second reactor was charged with compound V (the bis-HCl salt toluene solvate from Step 8) (39.4 g), toluene (206 g) and N,N-diisopropylethylamine (70.8 g) at 25° C. The content of reactor 1 was transferred to reactor 2 at 30° C. and rinsed with toluene (50 g). The mixture was stirred at 30° C. for another 0.5 h, then charged with isopropanol (84 g) and water (108 g) while maintained the temperature at 25° C. After stirring for 10 min, remove the aqueous phase after phase cutting. To the organic phase was charged isopropanol (43 g), water (54 g) and stirred for 10 min The aqueous phase was removed after phase cutting. The mixture was distilled under reduced pressure to remove ca. 250 mL of volatiles, followed by addition of methyl tert-butyl ether (MTBE, 238 g). The batch was stirred at 65° C. for over 1 hr, then cooled to 20 C over 1 hr and held for another 1 hr at the same temperature. The solid was filtered, rinsed with MTBE (95 g), and dried in vacuo at 80° C. to give (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate VII as a beige solid (50 g, 90% yield).

### Step 10. Synthesis of (E)-3-(2-(1-(2-(5-Bromopyrimidin-2-yl)-3-cyclopentyl-1-methyl-1H-indole-6-carboxamido)cyclobutyl)-1-methyl-1H-benzo[d]imidazol-6-yl)acrylic acid (Compound (1))

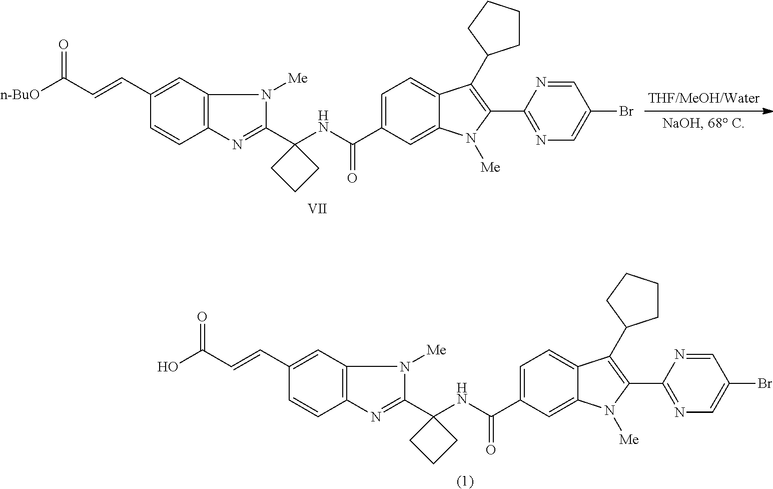

Notes:

In this process, hydrolysis of (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate was carried out in mixture of THF/MeOH and aq NaOH. Controlled acidification of the corresponding mesylate salt with acetic acid is very critical to obtain easy-filtering crystalline product in high yield and purity.

Procedure:

To the suspension of (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate VII (489.0 g, 91.9 Wt %, 633.3 mmol) in THF (1298 g) and MeOH (387 g) was added 50% NaOH (82.7 g, 949.9 mmol), followed by rinse with water (978 g). The mixture was stirred between 65-68° C. for about 1 h for complete hydrolysis. The resulting solution was cooled to 35° C., and filtered through an in-line filter (0.5 micron), and rinsed with a pre-mixed solution of water (978 g) and MeOH (387 g). The solution was heated to 60±4° C., and acetic acid (41.4 g, 689 mmol) was added over 1 h while the mixture was well agitated. The resulting suspension was stirred at 60±4° C. for 0.5 h. Another portion of acetic acid (41.4 g, 689 mmol) was charged in 0.5 h, and batch was stirred at 60±4° C. for additional 0.5 h. The batch was cooled to 26±4° C. over 1 h and held for 1 h. The batch was filtered, rinsed with a premixed solution of water (1956 g) and MeOH (773.6 g), dried at 50° C. under vacuum to give (E)-3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylic acid (1) (419.0 g, 95% yield) with ≧99.0 A % (240 nm) and 94.1 Wt % by HPLC.

### Step 11. Formation of Compound (1) Mesylate Salt

1. Mixed 6.0 g of Compound (1) free acid form (from Step 10) and 42 g ethyl acetate.
2. Heated the mixture to 60° C.
3. Added 0.18 g methanesulfonic acid (0.2 e.q.) to provide a resulting mixture which is a slurry. Stirred at 60° C. for ˜30 minutes.
4. Added 0.81 g methanesulfonic acid (0.9 e.q.) over 2 hours, then stir for 1 hour.
5. Cooled the slurry to 20° C. over 6 hrs.
6. Filtered the slurry and washed the wet cake with 6.0 g ethyl acetate.
7. Dried the wet cake at 60° C. under vacuum for at least 12 hours.

The crystalline material that was produced was subject to the XRPD and ssNMR analyses according to the analytical methods as described above. The resulting XRPD pattern shown in FIG. 1 and the ssNMR pattern shown in FIG. 2 confirm the formation of crystalline methanesulfonate (mesylate) salt of Compound (1).
1. The Compound (1) mesylate salt can also be prepared in MeCN or MEK instead of ethyl acetate with a similar procedure described above.
2. Other temperatures of salt formation may also be applied.

Pharmaceutical Formulations of the Crystalline Mesylate Salt

One class of solubilizer excipients, basifiers, act by increasing the microenvironment pH and thereby increasing the local solubility of the drug (for a drug containing an acid moiety). Another class of solubilizer excipients, surfactants, also act by increasing the local solubility of the drug. Formulations of the mesylate salt can be made using these excipients. Use of such solubilizers, such as basifiers and surfactants, with Compound (1) crystalline mesylate salt, would be expected to enhance drug solubility. Examples of surfactant excipients include, for example, sodium lauryl sulfate or Vitamin E TPGS, and examples of basifier excipients, which can be used separately or in combination with the surfactants are L-arginine, meglumine or L-lysine. These excipients can been applied in the development of solid oral tablet formulations. Other tablet formulation excipients, such as binders (which can also enhance solubilization), fillers, glidant and lubricants, to aid in the tabletting process, and disintegrants, to aid in tablet disintegration upon contact in aqueous media, may be added as required.

Thus, another embodiment of the present invention is directed to a solid pharmaceutical composition, e.g. a tablet, comprising:
(a) Compound (1) crystalline mesylate salt;
(b) at least one surfactant;
(c) at least one basifier;
(d) and optionally one or more pharmaceutically acceptable excipients, such as binders; fillers; glidants; and lubricants.

The amount of active ingredient of Compound (1) crystalline mesylate salt that may be present in the dosage form may vary widely or be adjusted depending upon the intended route of administration, the potency of the particular active ingredient being used, the severity of the hepatitis C viral infection and the required concentration. In a particular embodiment, the Compound (1) crystalline mesylate salt is present in a tablet-based formulation composition in an amount from about 1% to 90% by weight, preferably from about 5% to 50% by weight, more preferably from about 10% to 40% by weight.

Other pharmaceutically acceptable surfactants suitable for use in formulations of the Compound (1) crystalline mesylate salt include, but are not limited to, sodium lauryl sulfate (SDS), Vitamin E TPGS, Gelucire® or combinations thereof. A preferred surfactant is sodium lauryl sulfate. The surfactant can comprise 0% to 50% by weight of the total composition, with preferred amounts from 1-10% by weight of total composition and still more preferably from about 2% to 8% by weight of total composition.

Other pharmaceutically acceptable basifiers suitable for use in formulations of the Compound (1) crystalline mesylate salt include, but are not limited to, L-arginine, meglumine, L-lysine, tromethamine (Tris) or combinations thereof. A preferred basifier is L-arginine. The basifier can comprise 0% to 40% by weight of the total composition, with preferred amounts from 2-20% by weight of total composition and more still preferably from about 4% to 16% by weight of total composition.

The composition in accordance with any of the above-described embodiments optionally includes further excipients, such as tablet binders (e.g. water miscible polymers such as polyethylene glycols (different molecular weights) and polyvinyl pyrrolidone and water insoluble polymers such as copolymers of polyvinyl pyrrolidone and polyvinyl acetate, etc.), tablet fillers (such as microcrystalline cellulose, pharmaceutically acceptable sugars (such as lactose monohydrate, mannitol, isomalt, sorbitol, etc), glidants (such as talc, colloidal silicon dioxide, etc.), lubricants (such as magnesium stearate, etc). In this composition, the tablet binders such as polyethylene glycols (different molecular weights) also act as a solubilizer in the formulation and the composition preferable may contain such binder/solubilizer. Those of ordinary skill in the pharmaceutical art will know how to select acceptable binders, fillers, glidants and lubricants for tablet formulation.

Additionally, if in tablet form, the tablet may be film coated to form as a film coated tablet product using commonly known and commercially available film coating materials. Examples of film-forming polymers that can be used include polyvinyl acetate and hydroxypropyl methyl cellulose. These polymers are present in commercially available film coating systems such as OPADRY® I and OPADRY® II systems from Colorcon.

Additional preferred formulation embodiments include:

A solid pharmaceutical composition, e.g. a tablet, comprising:
(a) about 5 to 60% by weight Compound (1) crystalline mesylate salt;
(b) about 1 to 10% by weight surfactant;
(c) about 2 to 20% by weight basifier;
(d) 0 to about 40% by weight binder;
   and optionally one or more other pharmaceutically acceptable excipients

A solid pharmaceutical composition, e.g. a tablet, comprising:
(a) about 10 to 50% by weight Compound (1) crystalline mesylate salt;
(b) about 2 to 8% by weight surfactant;
(c) about 4 to 16% by weight basifier;
(d) about 1 to 25% by weight binder;
   and optionally one or more other pharmaceutically acceptable excipients.

A solid pharmaceutical composition, e.g. a tablet, comprising:
(a) about 20 to 50% by weight Compound (1) crystalline mesylate salt;
(b) about 2 to 6% by weight surfactant;
(c) about 4 to 12% by weight basifier;
(d) about 5 to 20% by weight binder;
   and optionally one or more other pharmaceutically acceptable excipients.

### Preparation of Formulations

The drug substance along with any intragranular excipients, for example, a basifier, surfactant, solubilizer/binder and/or filler if included, can be mixed in a dry state in a high shear granulator prior to addition of water. The drug substance and the excipients may be screened prior to milling to remove large agglomerates if necessary. After mixing is complete, the mixture is granulated using purified water as a granulating agent in the high shear granulator till a suitable end point is achieved. The wet granules are removed and dried at appropriate drying temperatures either in a tray dryer or a fluid bed dryer. The dried granules are milled by passing through a high speed mill, such as a Comill Milled granules are then blended with the extragranular excipients, including, for example, filler, glidant and lubricant and then tableted in a tablet press.

### Fluid Bed Granulation

Additional formulations can be prepared using a fluid bed granulation process instead of high shear granulation. Though the formulations are very similar to those prepared using high shear granulation, this fluid bed granulation process significantly reduces the manufacturing time and enables much easier and less challenging process scale-up compared to high shear granulation. These formulations may exhibit an advantage of significantly improved tableting properties (achieving target tablet hardness at significantly reduced compression forces during tableting operation) compared to the formulations manufactured using high shear granulation. The following general procedure can be used for fluid bed granulation formulations. An aqueous binder solution containing PEG 8000 (binder/solubilizer) or containing both PEG 8000 (binder/solubilizer) and arginine (basifier) is prepared first. The other intrangranular components, including active ingredient, and optionally surfactant, filler and/or basifier (optional, depending upon composition of the binder solution) are mixed in the dry state for ˜5 minutes in the fluid bed granulator to prepare the premixture. The premixture is maintained in a fluidized state and granulated by spraying the binder solution first followed by water into the fluid bed granulator, while adjusting process parameters such as product bed temperature, inlet air temperature, airflow rate, spray rate and atomization pressure as required. Drying of the granulation is continued by maintaining the granulation in a fluidized state at an elevated temperature until a desired end point of drying (loss on drying) is obtained. The dried granules are milled by passing through a high speed mill, such as a Comill Milled granules are then blended with the extragranular excipients, including filler, glidant and lubricant and then tableted in a tablet press. The core tablets obtained are further film coated using a standard film coating formulation, such as, Hydroxypropylmethyl cellulose-based standard OPADRY® or Polyvinylacohol-based OPADRY® II.

### Examples of Specific Embodiments of the Invention

1. A crystalline mesylate salt of the Compound (1) of the following formula:

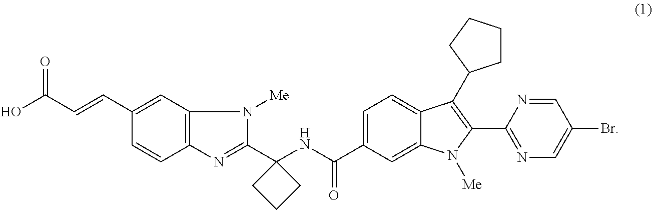 [SMILES: Cn1c(C2(NC(=O)c3ccc4c(C5CCCC5)c(-c5ncc(Br)cn5)n(C)c4c3)CCC2)nc2ccc(/C=C/C(=O)O)cc21]

2. A crystalline mesylate salt according to embodiment 1, which his anhydrous.

3. A crystalline mesylate salt according to embodiment for 2, having:
(a) an X-ray powder diffraction pattern comprising peaks at 9.2, 16.9, 21.8, and 23.6 degrees 2θ (±0.2 degrees 2θ) when measured using CuKα radiation; or
(b) a ¹³C solid state NMR spectrum comprising peaks at a chemical shift of 167.4, 114.2, and 19.9 ppm (±0.2 ppm); or
(c) both an X-ray powder diffraction pattern comprising peaks at 9.2, 16.9, 21.8, and 23.6 degrees 2θ (±0.2 degrees 2θ) when measured using CuKα radiation and a ¹³C solid state NMR spectrum comprising peaks at a chemical shift of 167.4, 114.2, and 19.9 ppm (±0.2 ppm); or
(d) an X-ray powder diffraction pattern comprising peaks at 9.2, 16.9, 21.8, and 23.6 degrees 2θ and at 6.7, 8.8, 9.6, 15.0, 16.6, 19.5 and 20.9 degrees 2θ (±0.2 degrees 2θ); or
(e) both an X-ray powder diffraction pattern comprising peaks at 9.2, 16.9, 21.8, and 23.6 degrees 2θ and at 6.7, 8.8, 9.6, 15.0, 16.6, 19.5 and 20.9 degrees 2θ (±0.2 degrees 2θ) and a ¹³C solid state NMR spectrum comprising peaks at a chemical shift of 167.4, 114.2, and 19.9 ppm (±0.2 ppm); or
(f) any of above embodiments (b), (c) or (e) wherein the ¹³C solid state NMR spectrum further comprises peaks at chemical shifts of 167.4, 114.2, 19.9 ppm and at 135.2, 112.0, 39.2, 28.4 ppm and, optionally, additionally at 133.5, 123.7, 35.7 ppm (±0.2 ppm).

4. The crystalline mesylate salt according to any of the preceding embodiments in substantially pure form.

5. A process for preparing a crystalline mesylate salt of the Compound (1) of any of the preceding embodiments, said process comprising the following steps:
(a) combining Compound (1) with a suitable solvent, such as ethyl acetate, MeCN, MEK or THF, and an aqueous methanesulfonic acid solution, with heating to a temperature of about 50-60° C.;
(b) stirring and optionally adding additional solvent to the mixture obtained in step (a) at about 50-60° C.;
(c) cooling the mixture obtained in step (b) to about 20-30° C., resulting in precipitation of Compound (1) mesylate salt crystals; and
(d) filtering with optional solvent wash and drying.

6. A crystalline mesylate salt prepared by the process according to embodiment 5.

7. A pharmaceutical composition comprising a crystalline mesylate salt of Compound (1) of any of the preceding embodiments and at least one pharmaceutically acceptable carrier or diluent.

8. The composition according to embodiment 7 further comprising a therapeutically effective amount of one or more other antiviral agents.

9. The composition according to embodiment 8, wherein said antiviral agent is selected from: ribavirin and amantadine.

10. The composition according to embodiment 8, wherein the antiviral agent is another anti-HCV agent.

11. The composition according to embodiment 8, wherein the other anti-HCV agent is an immunomodulatory agent selected from alpha-, beta-, delta-, gamma-, tau- and omega-interferon and pegylated forms thereof.

12. The composition according to embodiment 8, wherein the other anti-HCV agent is another inhibitor of HCV polymerase.

13. The composition according to embodiment 8, wherein the other anti-HCV agent is an inhibitor of HCV NS3 protease.

14. The composition according to embodiment 8, wherein the other anti-HCV agent is an inhibitor of another target in the HCV life cycle.

15. The composition according to embodiment 14, wherein said inhibitor of another target in the HCV life cycle is selected from an agent that inhibits a target selected from HCV helicase, HCV NS2/3 protease and HCV IRES, and an agent that interferes with the function of an NS5A protein.

16. A method of inhibiting the RNA-dependent RNA polymerase activity of the enzyme NS5B, encoded by HCV, comprising exposing the enzyme NS5B to an effective amount of a crystalline mesylate salt of the Compound (1) according to any of the preceding embodiments under conditions where the RNA-dependent RNA polymerase activity of the enzyme NS5B is inhibited.

17. A method of inhibiting HCV replication, comprising exposing a cell infected with HCV to an effective amount of a crystalline mesylate salt of the Compound (1) according to any of the preceding embodiments under conditions where replication of HCV is inhibited.

18. A method of treating HCV infection in a mammal, comprising administering to the mammal an effective amount of a crystalline mesylate salt of the Compound (1) according to any of the preceding embodiments, or a composition thereof.

19. A method of treating HCV infection in a mammal, comprising administering to the mammal an effective amount of a crystalline mesylate salt of the Compound (1) according to any of the preceding embodiments, or a composition thereof, in combination with one or more additional antiviral agents.

20. An article of manufacture comprising a composition effective to treat an HCV infection or to inhibit the NS5B polymerase of HCV and packaging material comprising a label which indicates that the composition can be used to treat infection by the hepatitis C virus, wherein said composition comprises a crystalline mesylate salt of the Compound (1) according to any of the preceding embodiments.

1. A crystalline mesylate salt of the Compound (1) of the following formula:

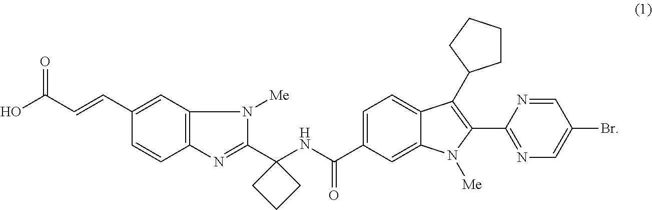 [SMILES: Cn1c(C2(NC(=O)c3ccc4c(C5CCCC5)c(-c5ncc(Br)cn5)n(C)c4c3)CCC2)nc2ccc(/C=C/C(=O)O)cc21]

2. A crystalline mesylate salt according to claim 1, which his anhydrous.

3. A crystalline mesylate salt according to claim 1, having:
(a) an X-ray powder diffraction pattern comprising peaks at 9.2, 16.9, 21.8, and 23.6 degrees 2θ (±0.2 degrees 2θ) when measured using CuKα radiation; or
(b) a ¹³C solid state NMR spectrum comprising peaks at a chemical shift of 167.4, 114.2, and 19.9 ppm (±0.2 ppm); or
(c) both an X-ray powder diffraction pattern comprising peaks at 9.2, 16.9, 21.8, and 23.6 degrees 2θ (±0.2 degrees 2θ) when measured using CuKα radiation and a ¹³C solid state NMR spectrum comprising peaks at a chemical shift of 167.4, 114.2, and 19.9 ppm (±0.2 ppm); or
(d) an X-ray powder diffraction pattern comprising peaks at 9.2, 16.9, 21.8, and 23.6 degrees 2θ and at 6.7, 8.8, 9.6, 15.0, 16.6, 19.5 and 20.9 degrees 2θ (±0.2 degrees 2θ); or
(e) both an X-ray powder diffraction pattern comprising peaks at 9.2, 16.9, 21.8, and 23.6 degrees 2θ and at 6.7, 8.8, 9.6, 15.0, 16.6, 19.5 and 20.9 degrees 2θ (±0.2 degrees 2θ) and a ¹³C solid state NMR spectrum comprising peaks at a chemical shift of 167.4, 114.2, and 19.9 ppm (±0.2 ppm); or
(f) any of above embodiments (b), (c) or (e) wherein the ¹³C solid state NMR spectrum further comprises peaks at chemical shifts of 167.4, 114.2, 19.9 ppm and at 135.2, 112.0, 39.2, 28.4 ppm and, optionally, additionally at 133.5, 123.7, 35.7 ppm (±0.2 ppm).

4. A crystalline mesylate salt according to claim 2, having:
(a) an X-ray powder diffraction pattern comprising peaks at 9.2, 16.9, 21.8, and 23.6 degrees 2θ (±0.2 degrees 2θ) when measured using CuKα radiation; or
(b) a ¹³C solid state NMR spectrum comprising peaks at a chemical shift of 167.4, 114.2, and 19.9 ppm (±0.2 ppm); or
(c) both an X-ray powder diffraction pattern comprising peaks at 9.2, 16.9, 21.8, and 23.6 degrees 2θ (±0.2 degrees 2θ) when measured using CuKα radiation and a ¹³C solid state NMR spectrum comprising peaks at a chemical shift of 167.4, 114.2, and 19.9 ppm (±0.2 ppm); or
(d) an X-ray powder diffraction pattern comprising peaks at 9.2, 16.9, 21.8, and 23.6 degrees 2θ and at 6.7, 8.8, 9.6, 15.0, 16.6, 19.5 and 20.9 degrees 2θ (±0.2 degrees 2θ); or
(e) both an X-ray powder diffraction pattern comprising peaks at 9.2, 16.9, 21.8, and 23.6 degrees 2θ and at 6.7, 8.8, 9.6, 15.0, 16.6, 19.5 and 20.9 degrees 2θ (±0.2 degrees 2θ) and a ¹³C solid state NMR spectrum comprising peaks at a chemical shift of 167.4, 114.2, and 19.9 ppm (±0.2 ppm); or
(f) any of above embodiments (b), (c) or (e) wherein the ¹³C solid state NMR spectrum further comprises peaks at chemical shifts of 167.4, 114.2, 19.9 ppm and at 135.2, 112.0, 39.2, 28.4 ppm and, optionally, additionally at 133.5, 123.7, 35.7 ppm (±0.2 ppm).

5. The crystalline mesylate salt according to claim 1 in substantially pure form.

6. A process for preparing a crystalline mesylate salt of the Compound (1) according to claim 1, said process comprising the following steps:
(e) combining Compound (1) with a suitable solvent and an aqueous methanesulfonic acid solution, with heating to a temperature of about 50-60° C.;
(f) stiffing and optionally adding additional solvent to the mixture obtained in step (a) at about 50-60° C.;
(g) cooling the mixture obtained in step (b) to about 20-30° C., resulting in precipitation of Compound (1) mesylate salt crystals; and
(h) filtering with optional solvent wash and drying.

7. A crystalline mesylate salt prepared by the process according to claim 6.

8. A pharmaceutical composition comprising a crystalline mesylate salt of Compound (1) according to claim 1 and at least one pharmaceutically acceptable carrier or diluent.

9. A pharmaceutical composition according to claim 8, wherein the crystalline mesylate salt is in substantially pure form.

10. A method of inhibiting the RNA-dependent RNA polymerase activity of the enzyme NS5B, encoded by HCV, comprising exposing the enzyme NS5B to an effective amount of a crystalline mesylate salt of the Compound (1) according to claim 1 under conditions where the RNA-dependent RNA polymerase activity of the enzyme NS5B is inhibited.

11. A method of inhibiting HCV replication, comprising exposing a cell infected with HCV to an effective amount of a crystalline mesylate salt of the Compound (1) according to claim 1 under conditions where replication of HCV is inhibited.

12. A method of treating HCV infection in a mammal, comprising administering to the mammal an effective amount of a crystalline mesylate salt of the Compound (1) according to claim 1, or a pharmaceutical composition thereof.

13. A method of treating HCV infection in a mammal, comprising administering to the mammal an effective amount of a crystalline mesylate salt of the Compound (1) according to claim 1, or a pharmaceutical composition thereof, in combination with one or more additional antiviral agents.
